(19) United States
(12) Patent Application Publication
(10) Pub. No.: US 2011/0082561 A1
(43) Pub. Date: Apr. 7, 2011

(54) SURGICAL IMPLANT

(76) Inventor: Perry Forrester, Houston, TX (US)

(21) Appl. No.: 12/897,547

(22) Filed: Oct. 4, 2010

Related U.S. Application Data
(60) Provisional application No. 61/248,711, filed on Oct. 5, 2009.

(51) Int. Cl. A61F 2/42

(52) U.S. Cl. 623/21.19

(57) ABSTRACT

A surgical implant having an elliptically-shaped base, a bottom surface, a peripheral rim and a top surface, the top surface having a concave portion. The stem extends from the bottom surface, and includes a core, a first wing portion extending laterally outwardly from the core and a second wing portion extending laterally outwardly from the core. Each of the first and second wing portions has a laterally outermost edge, said edge having at least one tooth portion projecting laterally outwardly. There is a cannulation extending through the base and the core of the stem.

### CROSS REFERENCE TO RELATED APPLICATION

This application claims the priority of U.S. Provisional Application No. 61/248,711 filed on Oct. 5, 2009, the disclosure of which is incorporated herein by reference for all purposes.

### BACKGROUND OF THE INVENTION

### Field of the Invention

The present invention relates to a surgical implant, and more particularly to a subtalor implant for use in correcting podiatric disorders.

### SUMMARY OF THE INVENTION

The present invention, in one aspect, provides an implant comprising a generally elliptically-shaped base, the base having a bottom surface, a peripheral rim and a top surface, the top surface having at least a portion which is concave. A tapered stem extends from the bottom surface of the base, and includes a core, a first wing portion extending laterally outwardly from the core, and a second wing portion extending laterally outwardly from the core. Each of the first and second wing portions has a laterally outermost edge, with at least one tooth and there is a cannulation extending through the base and the core of the stem.

### DESCRIPTION OF PRIOR ART

Many people suffer from metatarso-phalangeal (MPJ) complications. The prior art shows several plans for prosthesis to correct MPJ complications. While some cases require a total joint replacement, there are many cases where only one aspect of the joint as in knee replacement. In this case, a partial joint prosthesis with an articular surface is positioned partially within and adjacent to the resected bone at the joint in need of partial replacement. The articular surface of this partial joint prosthesis is designed to articulate within articular bone surface of the non-restricted bone in the joint.

U.S. Pat. No. 2,908,031, incorporated herein by reference for all purposes, discloses an implant wherein articulating surface configured to articulate with another one-piece component with a mating concave bone surface are against a mating convex bone surface. The implant has two conically-shaped stems for insertion into the intramedullary cap canals of the proximal talus and the metatarsal bone of the foot. U.S. Patent Publication 2007/0078518, incorporated herein by reference for all purposes, discloses a cannulated hemi-implant with a base and a threaded shank, the shank being cannulated and implantable within a mediallary canal of the talus or other bone in the foot.

### BRIEF DESCRIPTION OF THE DRAWINGS

FIG. 1 is the front elevation of view of one embodiment of the implant of the present invention;

FIG. 2 is a side elevation of view of the implant shown in FIG. 1;

FIG. 3 is a top, plan view of the implant shown in FIG. 1;

FIG. 4 is a bottom plan view of the implant shown in FIG. 1;

FIG. 5 is a cross-sectional view taken along the lines 4-4 of FIG. 4.

### DESCRIPTION OF PREFERRED EMBODIMENTS

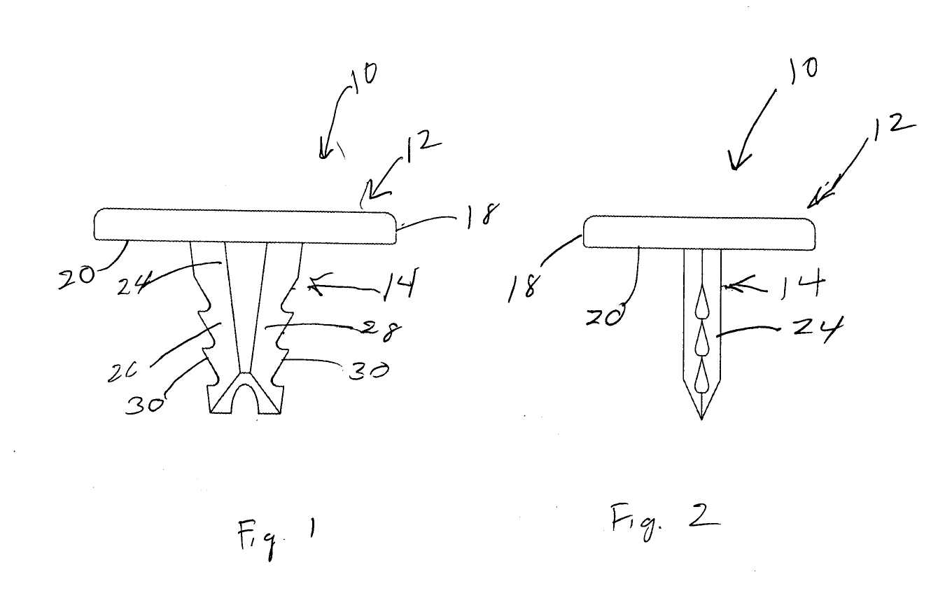

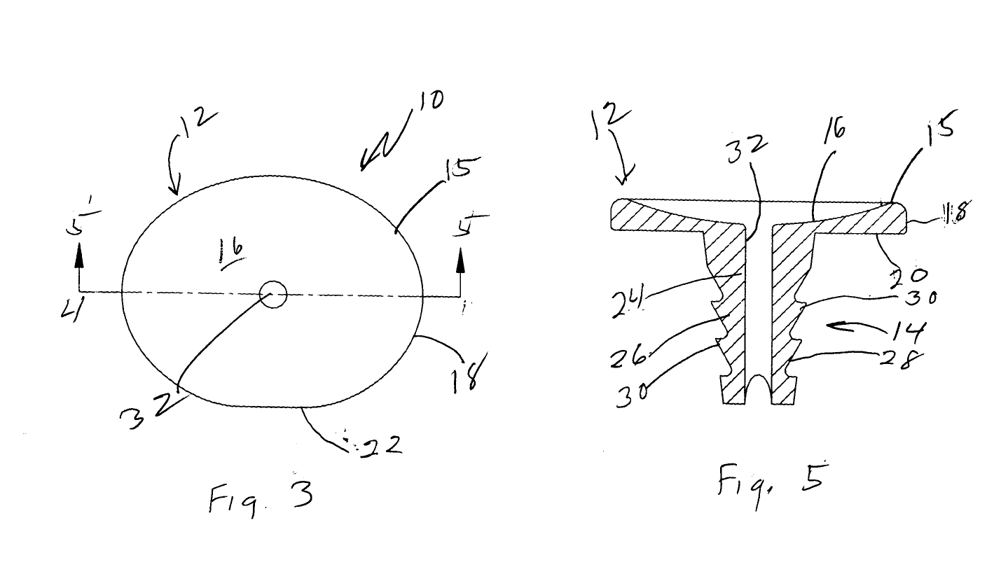

Referring first to FIGS. 1 and 2, the implant shown generally at 10 is comprised of a base, shown generally as 12 and a stem, shown generally as 14. As best shown in FIG. 5, base 12 has a top surface 15, a peripheral rim 18 and a bottom surface 20. As best seen with reference to FIG. 3, rim 18 of base 12 has a flat or substantially planar edge portion 22, which lies generally parallel to the long axis of the generally elliptically shaped base 12. As seen in FIG. 5 top surface 15 has a concave portion 16, while bottom surface 20 is substantially flat.

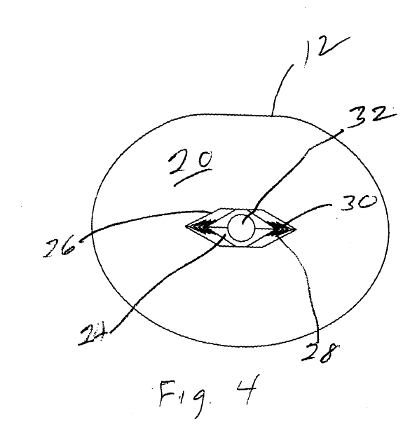

Stem 14 is comprised of a core portion 24 from which a first wing portion 26 extends laterally outwardly, there being a second wing portion 28 also extending laterally outwardly from core portion 24. While as shown, wing portions 26 and 28 are generally co-planar (see FIG. 4) in the sense that a plane lying within the long axis of the elliptical base 12 and perpendicular to the base 12 will pass through the wing portions 26 and 28, it will be understood that the wing portions could be at different angles to one another. Additionally, while only two wing portions are shown, it will be understood that stem 14 could include other wing portions as well. The laterally outer edges of wing portions 26 and 28 are serrated, having a plurality of teeth or hook-like formations 30 projecting laterally outwardly therefrom. While, as shown, there are plurality of such projections 30, it will be understood that the implant of the present invention can be made with only one tooth-like projection on each of the wing portions. A bore or cannulation 32 extends through base 12, generally centrally thereof, and the core portion 24 of stem 14.

The advantages of the surgical implant of the present invention are many. Tapered stem 14 is designed to match the shape of the medullary canal. Further, the generally hook-shaped form of the teeth 30 are designed for easy implantation but make the implant resistant to migration once it is implanted. Additionally, because of the relative thinness of stem 14, a minimum amount of cross-sectional area of the bone is impacted. Nevertheless, the unique design of the stem, with its hook-shaped teeth, firmly anchors the implant in the bone.

It is to be noted that all cannulated implants of the type under consideration are threaded. This can be undesirable as placement of the implant can be difficult, as is well known to those skilled in the art. However, the implant of the present invention requires no threading action, minimizing misalignment and the complications resulting therefrom when the implant is being used.

For example, two things are important when implanting an implant (1) the base of the implant must be firmly compressed against the face of the talus when it is implanted (2) the periphery of the base must conform to the extent possible with the periphery of the talus. When a threaded implant is used and when it is threaded sufficiently in to be firmly compressed against the face of the talus, it may be that the periphery of the base and periphery of the talus are not perfectly overlapping. One way to correct this is to back the threaded implant out some distance to cause the perforated base and the perforated talus to be superimposed upon one another. However, this is undesirable because then it leaves space between the base and face of the talus. Conversely, if to achieve alignment the threaded implant is further tightened, this may result in causing it to strip out leaving it loosely anchored to the bone. In other words, over-tightening the threaded implant is risky because it can strip out leaving it loosely anchored in the bone. Conversely, if the threaded implant is backed out all the way in order to reposition it, there remains the problem that when it is threaded back into the bone, it may ream out the prior bore made by the threads, again leaving it loose and/or misaligned.

Another disadvantage of a threaded implant is the fact that because it is basically a screw, the tapered threaded portion of the screw forms a relatively large cross-sectional area at the intersection with the underside or bottom surface of the base. It will be understood, as is well known to those skilled in the art, that dimensionally, the talus is generally oval shaped, like the base 12. The use of the implant of the present invention with the tapered stem 14 means that the widest portion of the stem can be aligned with the widest portion of the talus, while the narrower portion of the stem is aligned with the thinnest portion of the talus. This ensures that when the implant of the present invention is implanted, there is far less chance of breaking out of the bone.

As noted above, the implant of the present invention is cannulated. This serves an important function in the ease of implanting the implant and ensuring proper alignment. In implanting the implant of the present invention, an initial step, once the proper incisions have been made and the sizing of the implant (base) determined, is to insert a guide wire precisely in the desired location in the bone, which will serve to guide the implant in a proper direction with respect to the bone. This is accomplished by sliding the implant via the cannulation over the guide wire and then tapping the base to drive the implant into the bone. It should be noted and as pointed out above, that the taper of the stem closely matches the shape of the bone.

Another feature of the implant of the present invention is that the bottom surface of the base, as noted above, has a much greater area than the cross-sectional area of the stem at their point of juncture. This helps to ensure that the implant can be closely fitted to the face of the bone in which the implant is being positioned.

Also as noted above, the base 12 of the implant 10 and more specifically the peripheral rim 18 thereof has a flat, which generally lies parallel with the long axis of the generally elliptically-shaped base 12, and which permits proper accommodation of the flexor tendon.

Although specific embodiments of the invention have been described herein in some detail, this has been done solely for the purposes of explaining the various aspects of the invention, and is not intended to limit the scope of the invention as defined in the claims which follow. Those skilled in the art will understand that the embodiment shown and described is exemplary, and various other subimplanttutions, alterations and modifications, including but not limited to those design alternatives specifically discussed herein, may be made in the practice of the invention without departing from its scope.

What is claimed is:

1. A surgical implant comprising:
a generally elliptically-shaped base having a bottom surface, a peripheral rim and a top surface, said top surface having a concave portion; and
a stem extending from said bottom surface, said stern including a core, a first wing portion extending laterally outwardly from said core and a second wing portion extending laterally outwardly from said core, each of first and second wing portions having a laterally outermost edge, said edge having at least one tooth portion projecting laterally outwardly, a cannulation extending through said base and said core of said stem.

2. The implant of claim 1 wherein said peripheral rim has a flat, generally parallel with the long axis of the generally elliptical shaped base.

3. The implant of claim 1 wherein there are a plurality of said teeth.

4. The implant of claim 3 when both of said wings have a plurality of said teeth.

5. The implant of claim 1 wherein said stem is tapered, having its widest portion proximal said bottom surface, and its narrowest portion distal said bottom surface.

6. The implant of claim 1 wherein said first and second wings are generally co-planar.

7. The implant of claim 6 wherein said first and second wings lie generally in a plane passing through and perpendicular to said base.

8. The implant of claim 1 wherein said cannulation is substantially cylindrical.

9. The implant of claim 1 wherein said tooth is generally hook-shaped.
